(19) United States
(12) Patent Application Publication
(10) Pub. No.: US 2018/0353227 A1
(43) Pub. Date: Dec. 13, 2018

(54) ADJUSTABLE LENGTH ORTHOPEDIC DEVICE

(71) Applicant: Clarkson University, Potsdam, NY (US)

(72) Inventors: Laurel Kuxhaus, Potsdam, NY (US); Alexander Martin Clark, JR., Potsdam, NY (US); Stephen C. Palin, Jamesville, NY (US); Mark Hedgeland, Potsdam, NY (US); Deja Robinson, Potsdam, NY (US)

(73) Assignee: Clarkson University, Potsdam, NY (US)

(21) Appl. No.: 16/001,471

(22) Filed: Jun. 6, 2018

Related U.S. Application Data
(60) Provisional application No. 62/516,180, filed on Jun. 7, 2017.

(51) Int. Cl. A61B 17/74; A61B 17/72; A61B 90/00

(52) U.S. Cl. CPC A61B 17/742; A61B 17/72; A61B 2017/00477; A61B 90/08; A61B 2090/0811; A61B 90/03

(57) ABSTRACT

An adjustable length orthopedic device comprising: (i) a shaft having a longitudinal axis extending from a proximal end of the shaft to a distal end of the shaft, comprising an exterior having a diameter smaller than the medullary cavity of a bone within which at least a portion of the shaft is configured to reside, wherein the shaft comprises at least a portion of an adjustability mechanism located at said proximal end; (ii) a rod comprising a proximal end having at least a portion of an adjustability mechanism complementary to the adjustability mechanism of the shaft, wherein the adjustability mechanism of the shaft and the adjustability mechanism of the rod are configured to engage with one another in an adjustable manner; and (iii) a locking mechanism positioned at the intersection of the shaft and the rod configured to prevent rotation of at least one of the shaft and the rod.

### CROSS-REFERENCE TO RELATED APPLICATIONS

This application claims priority to U.S. Provisional Patent Application Ser. No. 62/516,180, filed on Jun. 7, 2017, and entitled “Developments on an Adjustable Length Orthopedic Device,” the entire contents of which are incorporated herein by reference.

### FIELD OF THE INVENTION

The present disclosure relates to an orthopedic device, and, more specifically, to an adjustable length orthopedic device.

### BACKGROUND

In the U.S., approximately 500,000 tibia fractures occur each year. Regardless of the fracture type or location within the tibia, intramedullary nails are chosen for fixation of the tibia between 82-87% of the time. In the tibia alone, an estimated 425,000 fractures a year are fixed with an intramedullary nail (sometimes called an intramedullary rod). The intramedullary rod or nail (“IMR” or “IMN”), also known as Küntscher nail, is a rod placed into the medullary cavity of a bone. These rods have been used to treat fractures of long bones of the body, including the tibia, femur, humerus, and others.

IM nails are often celebrated for their ability to help a patient return to an active lifestyle very quickly in comparison to other fracture fixation techniques such as external fixation or plating. While these repairs are typically successful, complications of malunion or nonunion occur in a significant number of tibial fractures treated with an intramedullary nail. These complications increase the healing time for patients and leads to higher rates of re-admittance, longer physical therapy, and more pain and discomfort for the affected patient. One contributing factor to these complications is the proper fit (that is, length) of the nail; a nail that is too long may cause nonunion or limb-length discrepancy; one that is too short may lead to periprosthetic fracture. Thus, improving the fit of the IM nail could reduce periprosthetic fracture and limb length discrepancies secondary to treatment of fractures with an IM nail.

Indeed, because of human size variability, there are differences in the length and width of the nails which typically requires the stocking of multiple length nails and may require left and right sided devices as well. Furthermore, the sizes are discrete (not continuous), which makes the fit approximate for a substantial proportion of the population. Post-operative complications of inserting an incorrectly-sized implant include fracture at the time of surgery or insufficient fixation.

The total monetary costs of IM nail use include those due to manufacturing (and unit cost), the costs necessary to maintain inventory (including turnover due to expiration of sterile packaging), the cost of insertion, and the costs of complication. Given that intramedullary nails are currently manufactured in discrete lengths, a hospital must stock more than one hundred sizes to ensure they have the right length ready for any patient needing the procedure, which is typically performed within hours of injury occurrence. Maintaining this inventory is costly to the healthcare system and can lead to waste (e.g., if a surgeon opens an improperly-sized nail, it must be discarded.) Patient outcomes could be improved, and IM nail manufacturing and complication costs reduced, with an adjustable-length intramedullary nail that would enable a patient-specific fit. Lag and locking screws are frequently used in fracture fixation and other orthopedic surgeries. Like the IMNs, these are manufactured in an array of discrete sizes, which may not be appropriate for any given patient. If the screws used are improperly-sized for the application, there can be intra-operative and post-operative complications. Using a screw that is too long can interfere with tendon gliding if the distal end of the screw exits the cortex; using a screw that is too short can create inadequate purchase into the cortex and thus not have sufficient structural integrity for healing. In either case, additional surgeries may be required to remedy.

Therefore, there is a continued need for fracture fixation devices that can be easily customized to give a patient-specific fit during orthopedic surgery.

### SUMMARY OF THE DISCLOSURE

The present disclosure is directed to an adjustable length orthopedic device configured to be easily customized to provide a patient-specific fit. According to an embodiment, the orthopedic device includes a shaft portion that fits within the medullary cavity of a bone. The device also includes a rod portion that also fits within the medullary cavity of a bone. The rod and shaft are configured to thread together, with each comprising threads and one configured to thread into or onto the other. By threading either the shaft or rod relative to the other, the length of the device is configurable. The device also includes a locking mechanism positioned at the intersection of the shaft and the rod. The locking mechanism prevents rotation of the shaft and/or the rod, which maintains the orthopedic device at the desired length.

According to one aspect is an adjustable length orthopedic device. The device comprises: (i) a shaft having a longitudinal axis extending from a proximal end of the shaft to a distal end of the shaft, the shaft comprising an exterior having a first diameter smaller than the diameter of a medullary cavity of a bone within which at least a portion of the shaft is configured to reside, wherein the shaft comprises at least a portion of an adjustability mechanism located at said proximal end; (ii) a rod comprising a proximal end having at least a portion of an adjustability mechanism complementary to the adjustability mechanism of the shaft, wherein the adjustability mechanism of the shaft and the adjustability mechanism of the rod are configured to engage with one another in an adjustable manner; and (iii) a locking mechanism positioned at the intersection of the shaft and the rod, wherein the locking mechanism is configured to prevent rotation of at least one of the shaft and the rod.

According to an embodiment, the shaft comprises at least one of a threaded interior cavity located at said proximal end and a threaded exterior region located at said proximal end, and wherein the proximal end comprises at least one of a threaded interior cavity and a threaded exterior region, wherein the proximal end of the rod is threaded into or onto the proximal end of the shaft.

According to an embodiment, the device is configured to reside within the medullary cavity of one or more bones.

According to an embodiment, the device is configured to transit a joint.

According to an embodiment, the locking mechanism comprises a locking nut. According to an embodiment, the locking nut is movably threaded onto the rod, and wherein a portion of the locking nut is configured to enter the interior cavity of the shaft as the rod is threaded into the shaft.

According to an embodiment, the locking mechanism further comprises a setscrew configured to affix the locking nut in place relative to the rod, or to affix the locking nut in place relative to the shaft. According to an embodiment, the setscrew is a flared setscrew and/or a threaded setscrew.

According to an embodiment, the locking nut is a collet. According to an embodiment, the collet comprises a tapered region configured to engage a portion of the shaft to interlock the shaft and the rod.

According to an embodiment, the locking mechanism comprises an interlocking mechanism. According to an embodiment, the interlocking mechanism comprises a first portion having a biased locking hook and a second portion comprising a locking tab, wherein the biased locking hook is configured to reversibly engage the locking tab to interlock the rod and the shaft.

According to an embodiment, the locking mechanism comprises two locking nuts.

According to an embodiment, the shaft comprises a pivoting locking tab configured to engage the threads of the rod as a locking nut threads onto a portion of the shaft and forces the locking tab to pivot.

According to an embodiment, the device is further configured to attach to another orthopedic device.

According to an embodiment, the device further includes a length indicator disposed along at least one of the shaft and the rod.

According to an embodiment, the device further includes at least one locking hole located in a distal end of the shaft.

According to an embodiment, the device further includes at least one locking hole located in a distal end of the rod.

According to an embodiment, the device is configured for use in an animal other than humans.

According to an embodiment, the locking mechanism is configured to prevent rotation of at least one of the shaft and the rod before the adjustable length orthopedic device is inserted within a bone.

According to an embodiment, the locking mechanism is internal to the rod and/or the shaft.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description are merely examples of the invention, and are intended to provide an overview or framework for understanding the nature and character of the invention as it is claimed. The accompanying drawings are included to provide a further understanding of the invention, and are incorporated in and constitute a part of this specification. The drawings illustrate various embodiments of the invention and together with the description serve to explain the principles and operation of the invention.

### BRIEF DESCRIPTION OF THE DRAWINGS

The present invention will be more fully understood and appreciated by reading the following Detailed Description in conjunction with the accompanying drawings, in which:

FIG. 1 is a schematic representation of an adjustable length orthopedic device with a locking mechanism, in accordance with to an embodiment.

FIG. 2A is a schematic representation of an adjustable length orthopedic device with a locking mechanism, at a first length, in accordance with to an embodiment.

FIG. 2B is a schematic representation of an adjustable length orthopedic device with a locking mechanism, at a second length, in accordance with to an embodiment.

FIG. 3 is a schematic representation of a locking mechanism of an adjustable length orthopedic device, in accordance with to an embodiment.

FIG. 4 is a schematic representation of a locking mechanism of an adjustable length orthopedic device, in accordance with to an embodiment.

FIG. 5 is a schematic representation of a locking mechanism of an adjustable length orthopedic device, in accordance with to an embodiment.

FIG. 6 is a schematic representation of a locking mechanism of an adjustable length orthopedic device, in accordance with to an embodiment.

FIG. 7 is a schematic representation of a locking mechanism of an adjustable length orthopedic device, in accordance with to an embodiment.

FIG. 8 is a schematic representation of a locking mechanism of an adjustable length orthopedic device, in accordance with to an embodiment.

FIG. 9 is a schematic representation of a locking mechanism of an adjustable length orthopedic device, in accordance with to an embodiment.

FIG. 10 is a schematic representation of screw for an adjustable length orthopedic device, in accordance with to an embodiment and a schematic representation of a locking screw positioned within an adjustable length orthopedic device and a bone, in accordance with to an embodiment.

FIG. 11 is a schematic representation of a locking mechanism of an adjustable length orthopedic device, in accordance with to an embodiment.

FIGS. 12A and 12B are schematic representations of a locking screw for an adjustable length orthopedic device, in accordance with to an embodiment.

FIG. 13 is a series of graphs of quasi-static stiffness for two adjustable length orthopedic devices, a prior art orthopedic device, and a human bone.

FIG. 14 is a graph of axial stiffness and torsional stiffness.

FIG. 15 is a schematic representation of an adjustable length orthopedic device, in accordance with to an embodiment.

FIG. 16 is a schematic representation of an adjustable length orthopedic device, in accordance with to an embodiment.

### DETAILED DESCRIPTION OF EMBODIMENTS

The present disclosure is directed to an adjustable length orthopedic device (“ALOD”) with the goal of decreasing inventory and improving patient outcomes by allowing adjustability of the implant to suit each patient. According to an embodiment, the ALOD comprises an adjustable orthopedic intramedullary nail as well as adjustable locking screws and lag screws. The ALOD described or otherwise envisioned herein revolutionizes intramedullary rods and orthopedic screws in a dramatic way to reduce costs for orthopedic implants. Among other improvements, the ALOD enables orthopedic implants to have length adjustment and locking which is currently not available with existing devices. The versatility of the device allows more patient-specific lengths of implants to be used for long bone fractures and many other uses and treatments, including but not limited to trauma, arthritis, infection, pain, growth disturbance, instability, tumors, cross-joint stability or treatment, and many others. Indeed, it will allow for orthopedic implants to have length adjustment that currently are not available.

According to an embodiment, the ALOD enables length adjustment of the device before insertion, once deployed in the patient, or both. The majority of intramedullary nails are between 300 and 400 cm. Therefore, having one device that may be adjusted in length for this range is highly beneficial compared to having multiple different sizes in that range to stock. According to an embodiment, the ALOD can be discretely adjusted and locked before it is implanted. This may be done with any of the locking mechanisms described or otherwise envisioned herein. As just one example, the locking mechanism may comprise a U-pin. For example, either the proximal or distal portion of the device may be the larger in diameter, with the U-pin as part of the smaller-diameter part. According to an embodiment, the ALOD is also be able to be locked in the patient with distal locking screws through the implant.

According to one embodiment, the ALOD may be used for different long bone fractures, including but not limited to the tibia, femur, and humerus. The ALOD nail comprises a design that can be scaled for use in all long bones or may be bone specific based on laterality, intramedullary width, or anatomy. The technology may ultimately be used for other long bones such as the radius, ulna, clavicle, fibula, metacarpals, or metatarsals. According to an embodiment, the ALOD may be utilized for other bone fractures as well as a wide variety of other treatments other than bone fractures.

According to an embodiment, the ALOD is equipped with a stepped shoulder screw, enabling contact with both cortices of the long bone. The adjustable screw length increases the accuracy of their length, allowing screw use in a locked or bicortical type fashion, and decreases inventory.

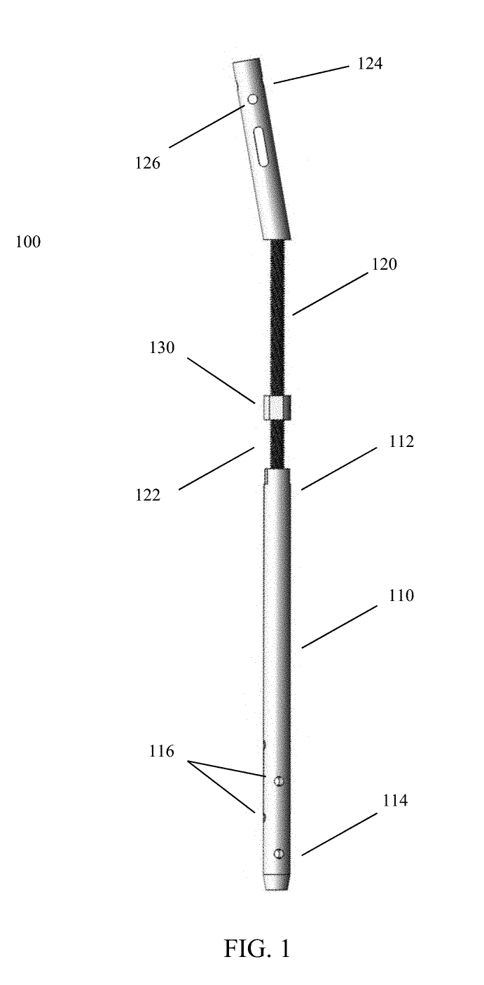

Depicted in FIG. 1 is a schematic representation of an adjustable length orthopedic device 100, in accordance with an embodiment. The adjustable length orthopedic device 100 comprises a shaft or bow 110 with a proximal end 112 and a distal end 114. The device also includes a rod 120 with a proximal end 122 and a distal end 124.

One or more of the proximal end 112 of the shaft and the proximal end 122 of the rod comprise an adjustability mechanism configured to convey adjustability of the length of the device, and/or to provide a mechanism to permanently or reversibly connect the shaft and the rod. For example, the adjustability mechanism may be a twist-lock, a slider with a pin, or pins like crutches. As another example, the adjustability mechanism may be complementary threading on the shaft and the rod. Many other examples of adjustability mechanisms are possible.

In the embodiment shown in FIG. 1, a proximal portion 122 of rod 120 is threaded as is an internal portion of the proximal end 112 of the shaft 110, and the rod is threaded into the shaft to provide adjustment of the length of the device. However, in another embodiment, an internal portion of the proximal end of the rod is threaded as is an external portion of the proximal end of shaft, and the shaft is threaded into the rod to provide adjustment of the length of the device. Both the shaft 110 and rod 120 can comprise a variety of shapes and sizes, including but not limited to a V-shape or slightly bent shape. For example, as shown in FIG. 1, the rod 120 comprises a slightly bent portion at the distal end 124, bent relative to the rest of the rod and the shaft. The angle may be 10°, for example, although angles greater than and less than 10° are possible. The depictions in the figures do not limit the potential sizes or shapes of the ALOD devices within the scope of the invention or the claims.

According to an embodiment, rod 120 may comprise a diameter less than the diameter of shaft 110, although many variations are possible. All or a portion of the exposed portion of rod 120 and/or shaft 110 can be smooth or can comprise protrusions or other components known in the art to engage bone surfaces. The threading of the rod 120 is complementary to the threading of the shaft 110 such that rotating either the rod or the shaft in a first direction adjusts the length of the device to be longer, while rotating either the rod or the shaft in a second direction adjusts the length of the device to be shorter.

According to an embodiment, device 100 comprises a locking mechanism 130 positioned at or near the intersection of the shaft and rod. The locking mechanism is configured to prevent rotation of at least one of the shaft and the rod, and thus may prevent rotation of both the shaft and the rod when the locking mechanism is actively locking the device. This prevents rotation, lengthening, and shortening of the device. Although shown on rod 120 in FIG. 1, it is recognized that the locking mechanism may be located on/in rod 120, on/in shaft 110, and/or on/in both rod 120 and shaft 110.

According to an embodiment, distal portion 124 of rod 120 comprises one or more receptacles, protrusions, or other gripping or receiving components that allows a screwdriver or other tool to engage or affix to the rod and either rotate the rod or hold the rod in place as the shaft is rotated, thereby elongating or shortening the length of device 100. Rotation (and thus the resulting length modification) can be performed before the orthopedic device is implanted, after it is implanted, or both.

According to an embodiment, distal portion 114 of shaft 110 comprises one or more receptacles, protrusions, or other gripping or receiving components that allows a screwdriver or other tool to engage or affix to the shaft and either rotate the shaft or hold the shaft in place as the rod is rotated, thereby elongating or shortening the length of device 100. Rotation (and thus the resulting length modification) can be performed before the orthopedic device is implanted, after it is implanted, or both.

According to an embodiment, distal portion 124 of rod 120 comprises one or more holes or openings 126 that allow for locking screws or lag screws to be inserted through the hole and into the bone. According to an embodiment, distal portion 114 of shaft 110 comprises one or more holes or openings 116 that allow for locking screws or lag screws to be inserted through the hole and into the bone. Similar to the shaft and rod of the ALOD, the length of locking screws and/or lag screws can be made adjustable to allow for the system to fit a variety of patients and bones. Adjustable length screws can be used for screws to be used that are unicortical or bicortical in their application. The ALOD screws can also be used for other orthopedic indications outside of their use with the ALOD rods for the treatment of fractures and other pathologies. Having adjustable screw lengths will increase the accuracy of their length, allow screw use in a locked or bicortical type fashion, and decrease inventory.

According to an embodiment, device 100 is used as a cephalomedullary nail to treat bone fractures, including but not limited to intertrochanteric and subtrochanteric fractures of the femur. According to this embodiment, a lag screw is placed through the device 100 (either shaft 110, rod 120, or both) and then inserted into the bone, such as the head of the femur. Typically, one or more screws are used to secure the ALOD in place and preserve proper length and alignment.

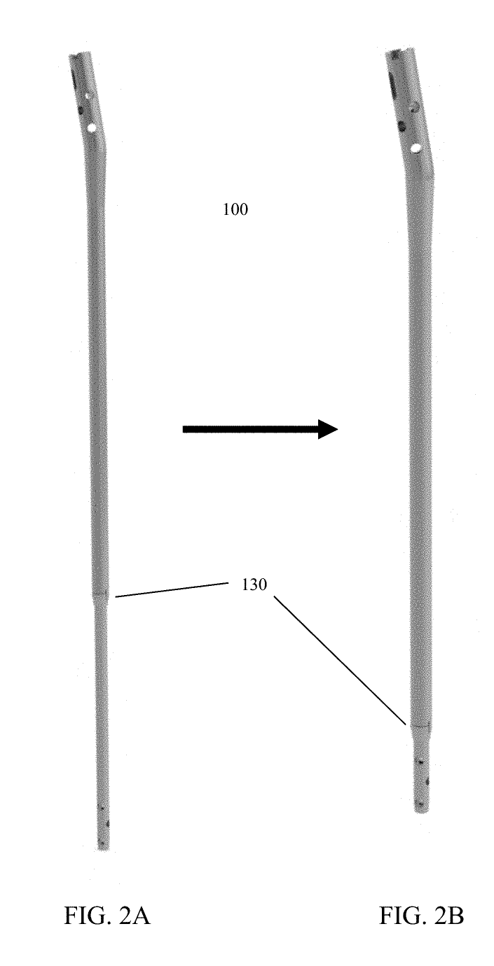

Referring to FIG. 2A is an embodiment of an adjustable length orthopedic device 100 in an extended state, and referring to FIG. 2B is an embodiment of the same adjustable length orthopedic device 100 in a shortened state. Notably, the images are not to scale, as the configuration of the device in FIG. 2B will be significantly shorter than the configuration of the device in FIG. 2B. The rod and/or shaft of device in FIG. 2A has been rotated or otherwise shortened to reduce the length of the device 100. Device 100 in FIGS. 2A and 2B comprise a locking mechanism 130.

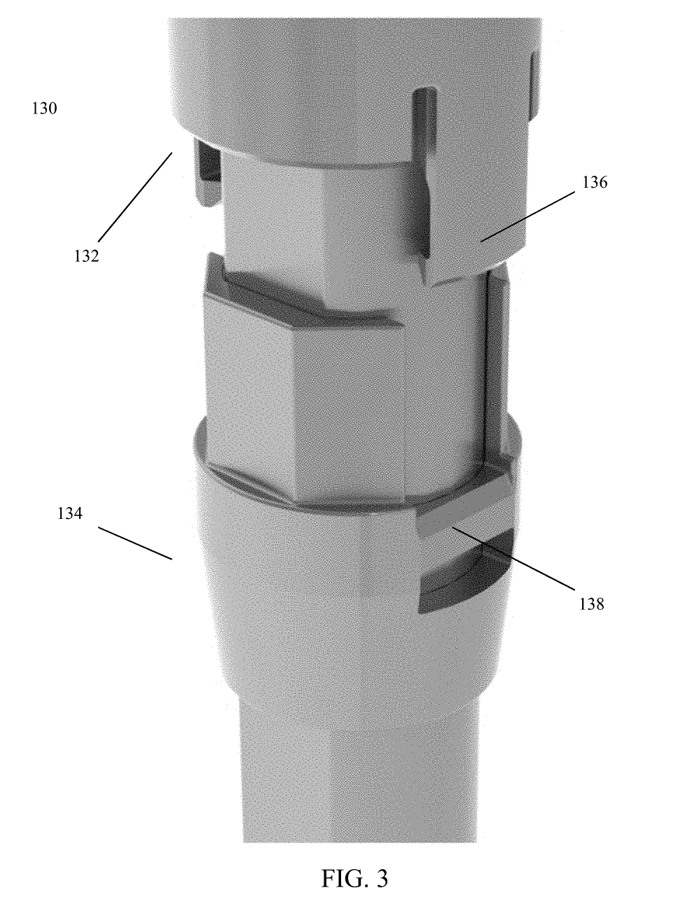

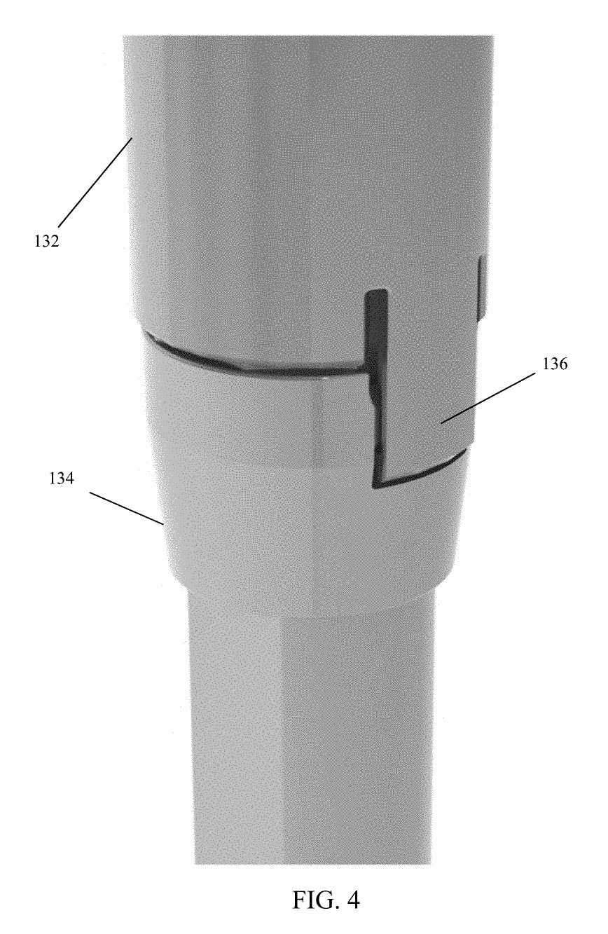

Referring to FIGS. 3 and 4 is an embodiment of a locking mechanism 130 of, for example, FIGS. 2A and 2B. The locking mechanism 130 is an interlocking locking mechanism, comprising a first portion 132 on the shaft 110 and a second portion 134 on the rod 120, although the first and second portions may be reversed. In this embodiment, when first portion 132 and second portion 134 are interlocked, the rod and shaft are locked and thus the device is a fixed length. According to an embodiment, first portion 132 comprises a biased locking hook 136, which is biased inward toward the center of the rod 120. Second portion 134 comprises a locking tab 138. When the locking mechanism is locked, force is applied to first portion 132 and/or second portion 134 to force the locking hook 136 over the locking tab 138, such that the lip of the locking hook snaps into a locking tab space of the second portion 134 to hold the first portion and the second portion firmly interlocked, as shown in FIG. 2.

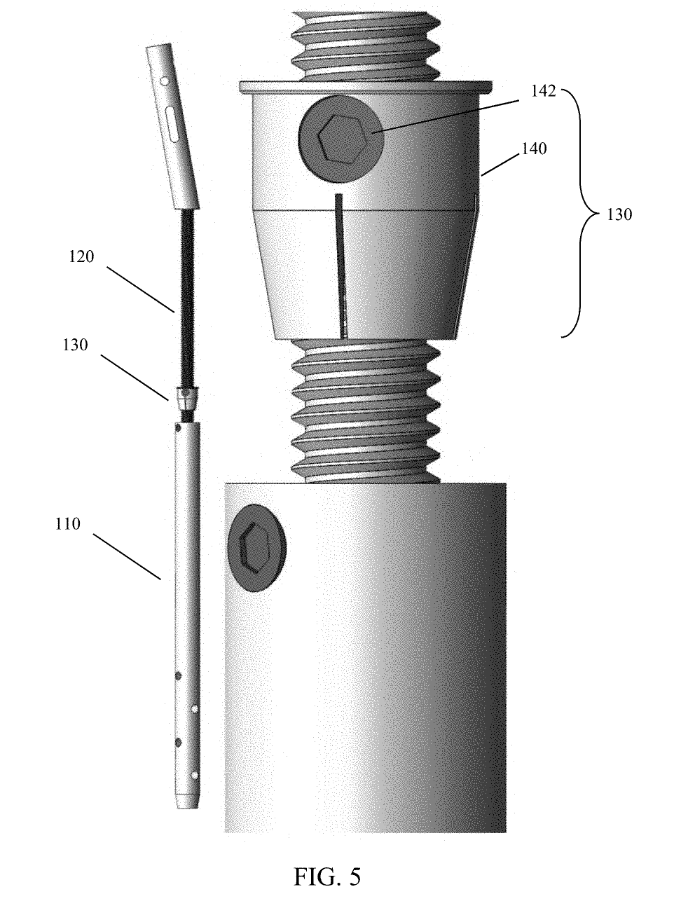

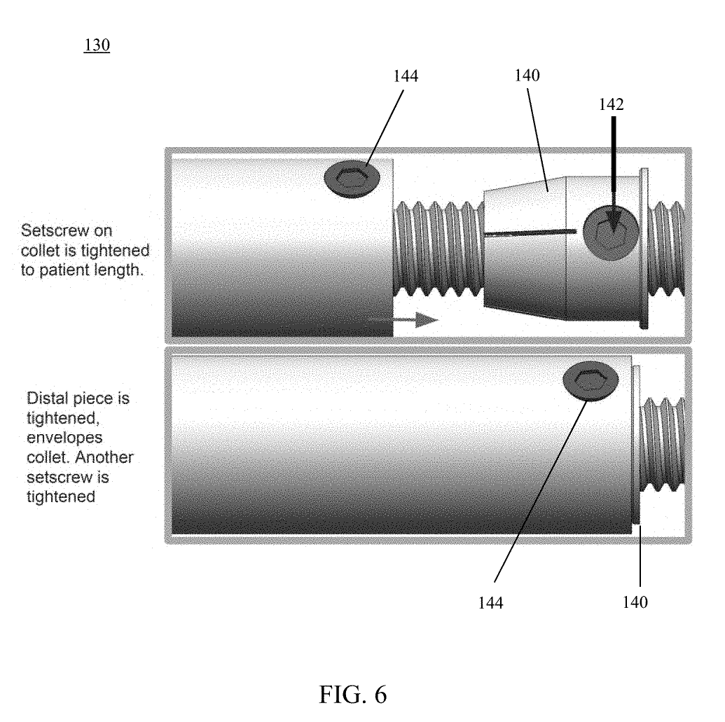

Referring to FIGS. 5 and 6, in one embodiment, is a locking mechanism 130. The locking mechanism 130 comprises a collet 140 threaded or otherwise positioned on rod 120 (although in another embodiment the collet 140 may be threaded or otherwise positioned on shaft 110). A setscrew 142 may be positioned on and/or in collet 140 to firmly affix the collet at a desired position along the rod 120. When the device is set at a desired length, the setscrew is tightened to hold the collet in place. The rod 120 can then be threaded onto/into shaft 110 until the collet interacts with a complementary portion of shaft 110, thereby holding the device firmly in place to prevent rotation, lengthening, and shortening, as shown in FIG. 6. According to an embodiment, the complementary portion of shaft 110 may comprise a setscrew 144 which can be tightened to hold the collet 140 firmly in place, further preventing rotation, lengthening, and shortening of the device.

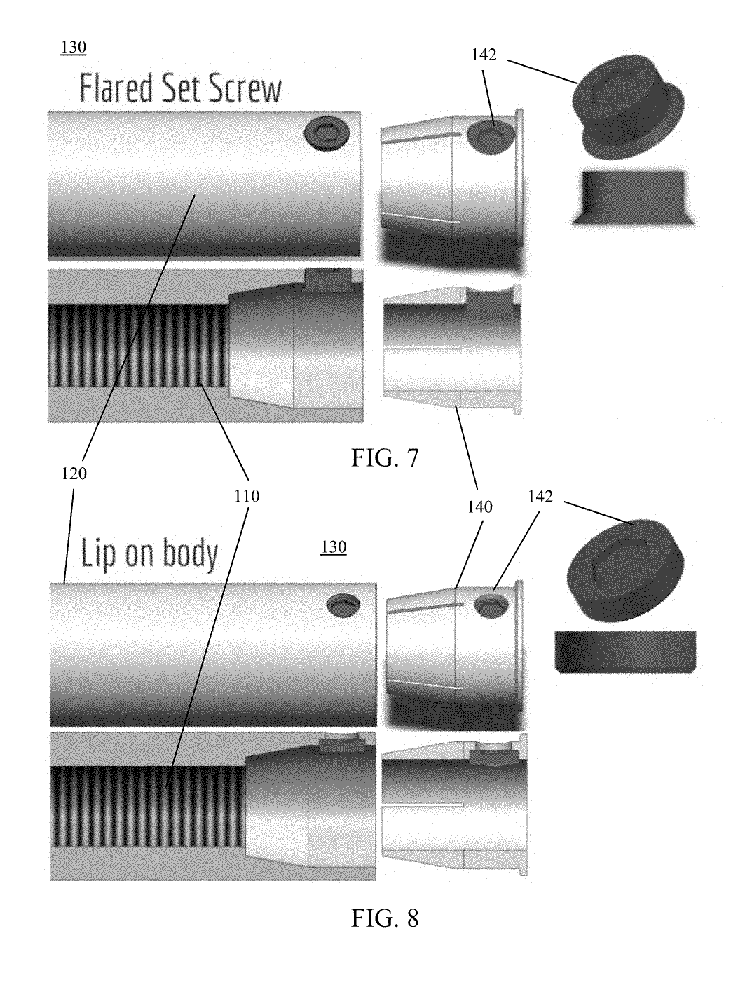

Referring to FIGS. 7 and 8 are embodiments of locking mechanism 130. In this embodiment, the locking mechanism 130 comprises a collet 140 on rod 120 with a setscrew 142. The shaft 120 optionally comprises a second setscrew as shown in the figures. The setscrew 142 is configured to tighten the collet onto the rod 120, as shown in the cross-section of collet 140 in FIG. 7. In FIG. 7, the setscrew 142 is flared and may comprise threads, although the threads are not shown. According to one embodiment, the setscrew of FIG. 7 may comprise measurements such as a thread length of 1.25 mm, a screw length of 1.75 mm, and a surface area of 12.50 mm², although many other measurements are possible. In FIG. 8, the setscrew 142 is not flared and may comprise threads, although the threads are not shown. The setscrew 142 is configured to tighten the collet onto the rod 120, as shown in the cross-section of collet 140 in FIG. 8. According to one embodiment, the setscrew of FIG. 8 may comprise measurements such as a thread length of 1.25 mm, lip of 0.5 mm, a screw length of 1.75 mm, and a surface area of 9.62 mm², although many other measurements are possible.

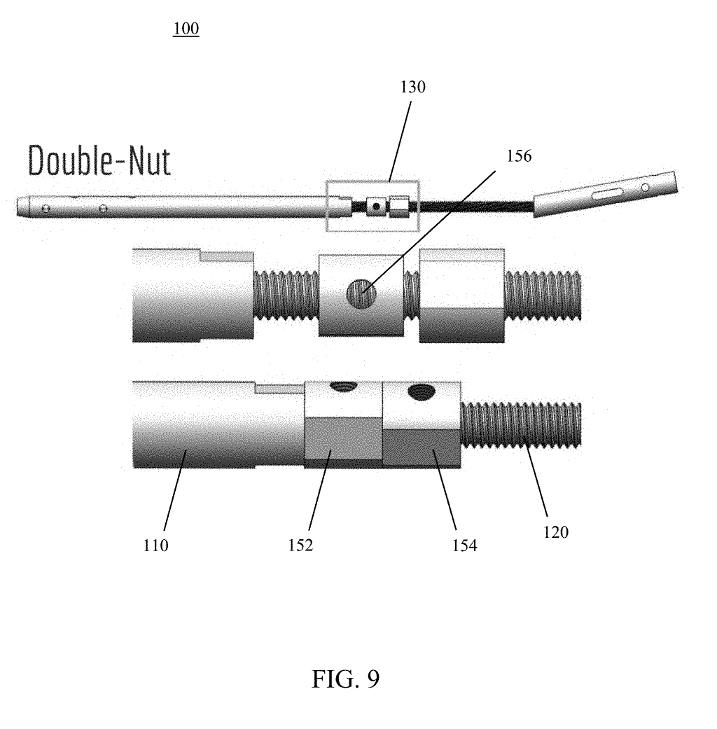

Referring to FIG. 9 is an embodiment of is a locking mechanism 130. The locking mechanism 130 comprises a double-nut system with a first nut 152 and a second nut 154. The first and second nuts can be threaded to thread on one or more of shaft 110 and rod 120, and thus comprise internal threading complementary to the threads of one or more of shaft 110 and rod 120. In FIG. 9, the first and second nuts are threaded on rod 120. To lock the device, one or more of the first and second nuts can be tightened onto rod 120 using a screw, setscrew, or other mechanism (not shown in FIG. 9) via opening 156 in one or more of nut 152 and nut 154. With the first and/or second nuts firmly positioned on rod 120, the rod will lock into place as it is threaded into or onto shaft 110, as shown in the bottom of FIG. 9.

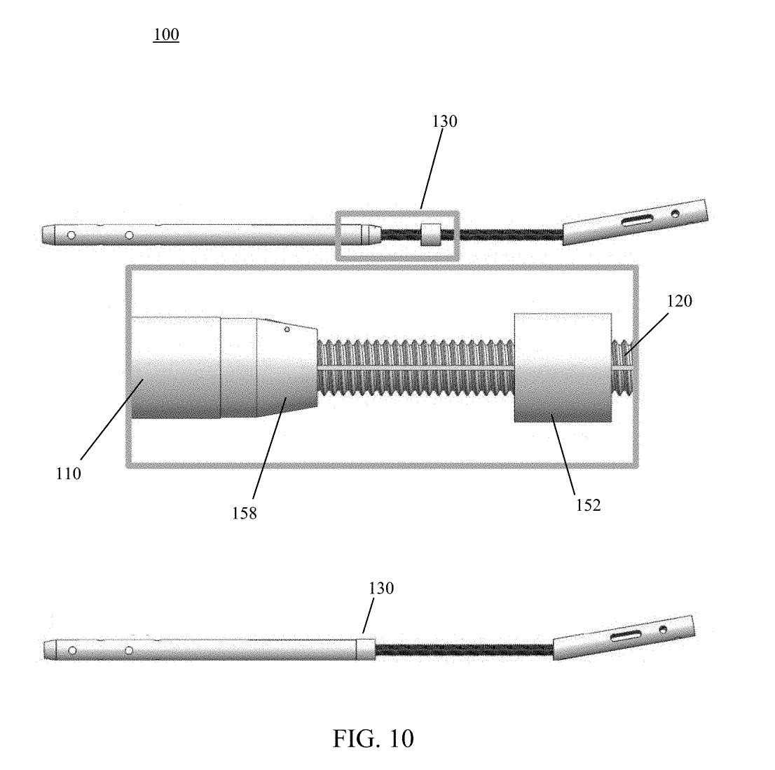

Referring to FIG. 10 is an embodiment of is a locking mechanism 130. The locking mechanism 130 comprises a single nut 152 which may be affixed or threaded onto the rod (or, in other embodiments, on the shaft 110). To lock the device, nut 152 can be tightened onto rod 120 using a screw, setscrew, or other mechanism (not shown in FIG. 10), which holds the nut firmly in place on the rod. As the rod is screwed into or onto shaft 110, the nut 152 encounters a tapered region 158 of the proximal end of shaft 110. Although not show in FIG. 10, nut 152 comprises a complementary tapered region that is configured to reversibly engage the tapered region 158 of shaft 110. When the tapered region of nut 152 is fully engaged with the tapered region 158 of shaft 110, the locking mechanism 130 is locked, preventing rotation, lengthening, and shortening, as shown in the bottom panel of FIG. 10.

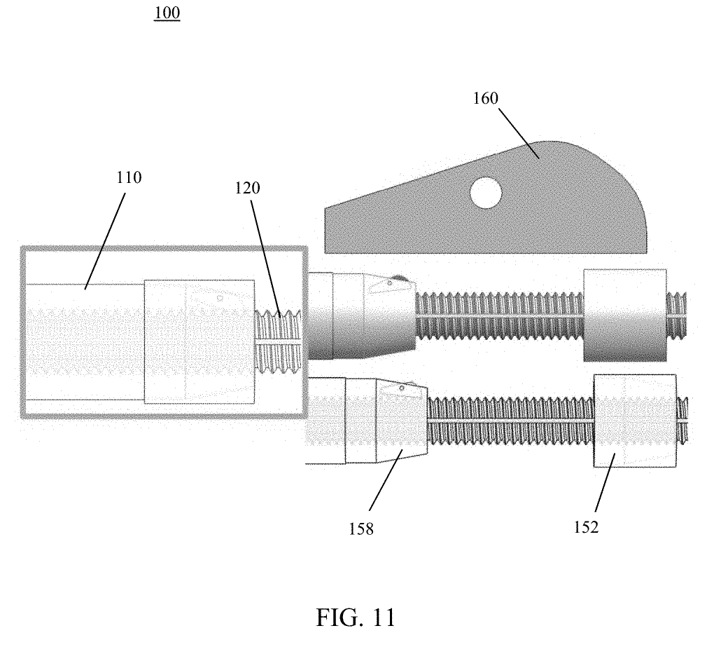

Referring to FIG. 11 is an embodiment of is a locking mechanism 130. The locking mechanism 130 comprises a pivoting locking tab 160 configured to engage the threads of the rod 120 as a locking nut 152 threads onto a portion of the shaft and forces the locking tab to pivot into the threads of the rod. To lock the device, nut 152 can be tightened onto rod 120 using a screw, setscrew, or other mechanism (not shown in FIG. 11), which holds the nut firmly in place on the rod. As the rod is screwed into or onto shaft 110, the nut 152 encounters a tapered region 158 of the proximal end of shaft 110. Although not show in FIG. 11, nut 152 comprises a complementary tapered region that is configured to reversibly engage the tapered region 158 of shaft 110. As the tapered region of nut 152 engages the tapered region 158 of the proximal end of shaft 110, it pushes on the pivoting locking tab 160 to force the locking tab to pivot into the threads of the rod, as shown in the left-hand panel of FIG. 11.

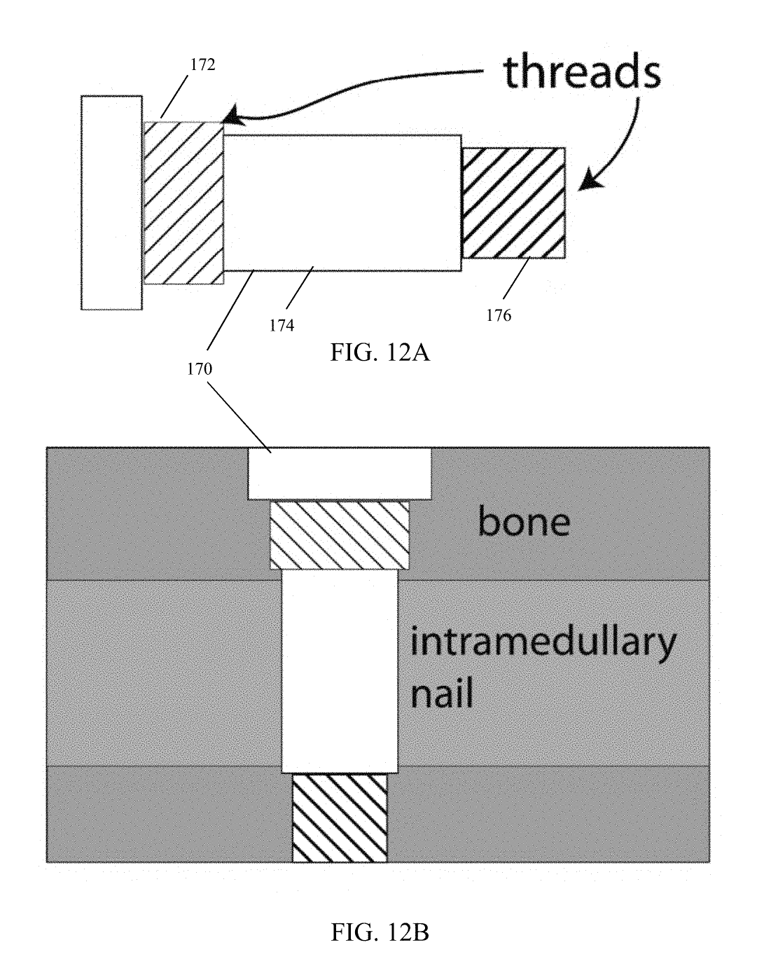

Referring to FIGS. 12A and 12B, the device is equipped with a stepped shoulder screw, enabling contact with both cortices of the long bone. The adjustable screw length increases the accuracy of their length, allowing screw use in a locked or bicortical type fashion, and decreases inventory. In FIG. 12A is a stepped screw 170 with a first threaded section 172 configured to engage one side or portion of a bone, a middle non-threaded section 174 configured to engage or interact with the ALOD, and a second threaded section 176 configured to engage a second side or portion of the bone.

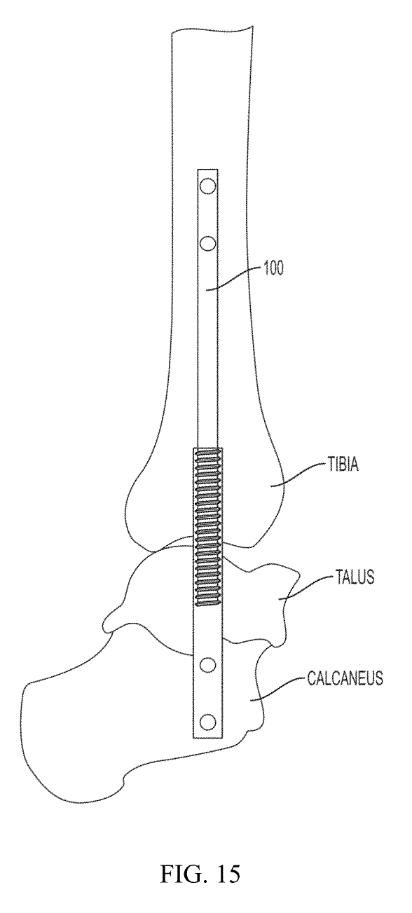

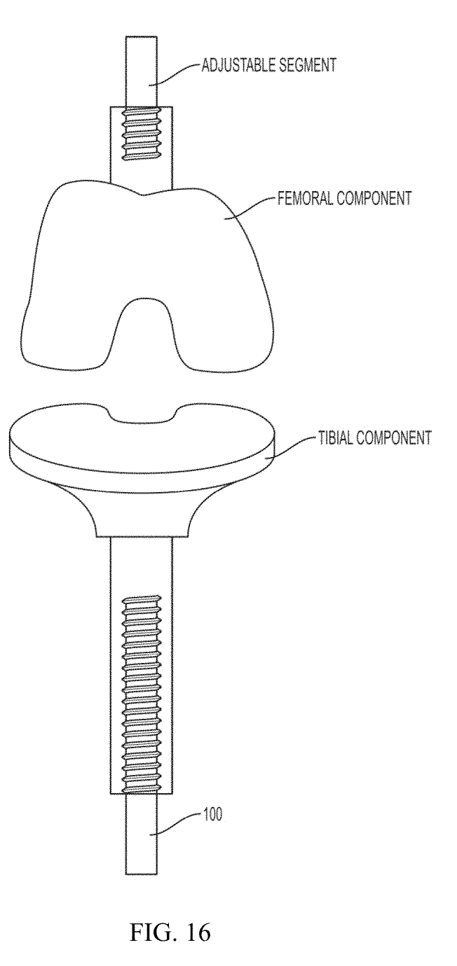

According to an embodiment, the device may be configured to transit a joint and/or one or more bones. Referring to FIGS. 15 and 16, for example, the adjustable length device is configured to transit one or more joints, such as the knee or the foot among many other joints or similar locations.

Example Analysis of One Embodiment of an Adjustable-Length Intramedullary Nail

According to an embodiment is an adjustable-length intramedullary nail as described or otherwise envisioned herein, which is configured to reduce both complications secondary to fracture fixation and manufacturing costs. Although it will be recognized that the example provided below is an example embodiment and does not limit the scope of the embodiments described or otherwise envisioned herein, it is provided to show one or more benefits of the adjustable-length intramedullary nail.

According to an embodiment, three prototypes of an adjustable-length intramedullary nail were manufactured and evaluated in quasi-static axial compression and torsion and quasi-static 4-point bending. Prototypes were dynamically evaluated in both cyclic axial loading and 4-point bending, and torsion to failure. The prototypes exceeded expectations. They were comparable in both quasi-static axial stiffness (1.41±0.37 N/min cervine tibiae and 2.30±0.63 in cadaver tibiae) and torsional stiffness (1.05±0.26 Nm/degree in cervine tibiae) to currently-used nails. The quasi-static 4-point bending stiffness was 80.11±09.360, greater than reported for currently-used nails. A length-variance analysis indicates that moderate changes in length do not unacceptably alter bone-implant axial stiffness. After 103,000 cycles of axial loading, the prototype failed at the locking screws, comparable to locking screw failures seen clinically. The prototypes survived 1,000,000 cycles of 4-point bend cyclic loading, as indicated by a consistent phase angle throughout cyclic loading. The torsion-to-failure test suggests that the prototype has adequate resistance to applied torques that might occur during the healing process.

Methods

Design and Specifications

To ensure the nail's functionality and interface with surgical instrumentation, design criteria were established based on currently-used nails. These criteria included that the nail must: (1) have a 10° degree proximal bend for ease of insertion; (2) be cannulated to accept a 3 mm guidewire, as required by the insertion process; (3) have at least 3 distal locking holes, including at least one each in the AP (Anterior-Posterior) & ML (Medial-Lateral) directions; (4) have two proximal locking holes, one each in the AP & ML directions; (5) have one dynamic locking hole (slot) at the proximal end; (6) adjust within the range of commonly available IM nail lengths (210-425 mm), ideally covering at least 50% of this range; and (7) be made of either surgical stainless steel, 316L (in early stages) or Ti6AI4V (later stages). For physician acceptance, the nail should have a similar appearance to currently-used nails. Following design, prototypes were manufactured and mechanically evaluated as described below.

Notably, these criteria do not limit the scope of the present embodiments and application. Rather, they are specific embodiments utilized for a specific study of one particular embodiment of the adjustable-length intramedullary nail.

Design and Specifications

According to the embodiments utilized in this example, each prototype comprised a bent proximal end welded to a threaded rod which inserts into a distal housing end with a female thread, allowing for adjustability at the mid-shaft of the prototype. The desired length is set by extending or retracting the proximal end out of or into the distal housing, and then locked in place using a combination locking nut with set screw which tightens against the distal housing. This design met all design criteria detailed above. Two copies of the prototype was manufactured in surgical stainless steel (316 L) at the Clarkson University machine shop, and in a titanium alloy (Ti6AI4V) at a third party manufacturing location (Incodema Inc., Ithaca, N.Y.) for evaluation as described below. Due to manufacturing challenges, the distal housing of the titanium alloy prototype was made using a welded threaded insert, providing 38.1 mm of thread, in contrast to the stainless steel version which had approximately 100 mm of tapped thread. This offered clearance for the 7 mm proximal end at the rest of the depth.

Mechanical Testing

To evaluate the fixation and locking of the adjustable-length IM nail, both quasi-static and cyclic loading tests were performed. Quasi-static axial, torsional, and 4-point bending stiffness was measured for the manufactured prototypes, as described below. Then, the prototype was subjected to 4-point bending and axial cyclic loading, and ultimate failure in torsion. In both axial and torsional testing, the prototype was inserted into a cadaveric tibia; 4-point bending tests were performed on the bare metal prototype.

Specimen Preparation (Axial and Torsional Testing)

Ten total cadaver deer tibiae (age 2.1±0.9 years, 5 male and 5 female) and 7 total cadaver human tibiae (age 74.8±9.2, 4 male and 3 female) were used. Cervine tibia specimens were obtained from a local meat provider, and human tibiae from Medcure (Portland, Oreg.). No specimens had visibly observable deformities or bony pathologies. Insertions were performed in the laboratory following a modified surgical procedure and under the advisement of a Board-Certified Orthopaedic Surgeon. First, the intramedullary canal was reamed using stainless steel flexible reamers (range: 9 mm to 12 mm in diameter). After reaming, midshaft fractures were simulated by transecting the tibia with a hacksaw at 50% of its total measured length. The appropriate nail length was measured using a guide wire. The wire was inserted to the distal end of the medullary canal, and then marked at its junction with the anterior edge of the tibial plateau; this section was then held next to a flexible tape measure and appropriate nail length was determined to the nearest millimeter. The prototype was then adjusted to the appropriate length prior to insertion. This length, and the total length of the tibia were recorded. After insertion, the prototype was locked in place at both ends using four (2 proximal and 2 distal) stainless steel round-head screws, size M6×1 (Length=38.1 mm). All tibia-nail constructs were potted at both ends in PVC cups filled with auto body filler (Bondo, 3M Corporation, Maplewood, Minn.) to ensure proper alignment. Throughout preparation and testing, specimens were kept moist with physiologic saline (0.159 M) applied periodically.

Quasi-Static Testing

Tests were conducted in an MTS axial/torsional hydraulic load frame (MTS 809 Axial/Torsional Test System, Eden Prairie, Minn.) Each of the quasi-static specimens (10 cervine and 5 human cadaver tibiae) were tested in both axial and torsional loading with the same stainless steel prototype. The subset of the total human cadaver specimens used for these quasi-static tests included 3 male (2 female) with an average age of 75.5±9.7. Load and displacement data were recorded at 100 Hz. Stiffness in each mode was calculated from the load-displacement data using a custom MATLAB (The MathWorks, Natick, Mass.) code. All stiffness testing and analysis was performed as specified in ASTM F1264. Quasi-static axial compression was done at 0.1 mm/sec, to a maximum compression increasing from 1 mm to 5 mm; the nail was unloaded in between each test. Axial stiffness, KA, was computed from the 5 mm trial using Equation 1:

K_{A}=ΔF/Δδ (Eq. 1)

where F is force in Newtons, and δ is displacement in mm. The axial compression was performed three times and the average of the three trials was used in analysis.

Torsional testing was performed at a speed of 0.1 degrees/sec; torsion was applied and the specimen was returned to neutral. This occurred in 1 degree increments, from 1 to 5 degrees, and torsional stiffness, KT, was computed per ASTM 1264 as in Equation 2:

K_{T}=ΔM/Δθ (Eq. 2)

where M is Torque in Nm and θ is angle in degrees. The complete torsion test was performed three times per specimen. The maximum stiffness was used in analysis.

Four-point bending tests were performed on the same stainless steel prototype at 0.1 mm/sec at 1 mm increments of compression in a fixture with the following dimensions: L=228 mm, s=c=76 mm, r=1 cm. Bending stiffness, K_{Bending}, was computed using Equation 3:

$${K}_{Bending} = \frac{( L + 2 C ) ( Δ F / Δδ ) {s}^{2}}{12} \qquad ( Eq . 3 )$$

where L, s, and c are based on roller geometry. Five trials were performed on each prototype, and the results averaged. Stiffness was then compared to that of previous adjustable-length prototypes, currently in-use discretely sized nails, and intact human and cervine cadaver tibiae.

Axial and Bending Cyclic Loading

The prototypes were dynamically evaluated in both axial and bending cyclic loading. The second copy of the stainless steel prototype was tested in both axial and 4-point bending cyclic loading; the titanium prototype was evaluated only in bending cyclic loading as described below. Data for each cyclic loading test was collected at 100 Hz on the axial/torsional hydraulic load frame described above. One human tibia specimen (62, M) with the nail inserted was tested in axial cyclic loading. The tibia/stainless steel prototype construct was cycled at a physiological load, between 2400 N and 100 N of compression at 3 Hz of sinusoidal loading to a maximum of 250,000 cycles was reached. The load-displacement measurements were then used to compute the phase angle during the cyclic loading was computed every 10,000 cycles as:

$$ϕ = \frac{2 π ( Δ {T}_{peak} )}{T} \qquad ( Eq . 4 )$$

where Δtₚₑₐₖ is the time difference between peak stress and peak strain (the same as the difference between peak load and peak displacement), and T is the period of loading (0.2 seconds for the loading scenario.)

4-point bending cyclic loading was also performed on the bare prototypes. This bending load cycling was performed on both a stainless steel and a titanium alloy prototype. The prototype was cyclically compressed in the fixture under physiologically-relevant loading conditions: between 500 N and 50 N, at a frequency of 5 Hz of sinusoidal loading, as outlined in ASTM F1264. Cycling continued until visual and/or aural failure was observed or 1,000,000 cycles was reached. The phase angle (described above) was then computed every 100,000 cycles.

Torsional Failure

The titanium alloy prototype was tested, in a cadaver tibia (68, M), to monotonic failure in torsion. First the tibia/prototype construct was torqued at rate of 1 degree/sec in external rotation until 60° of rotation or auditory and/or aural failure was observed. For this specimen, the external direction of rotation corresponds to loosening of the locking nut (on left tibia specimens, and corresponds to tightening in right tibiae). Regardless of performance, the constructs were returned to center (no torque conditions; zero degrees of rotation), and internally rotated at the same rate. Prototypes were then removed from the tibiae and visually inspected to ascertain the method of failure. The ultimate torsional strength in each direction was computed.

Additional Analysis

A length variance analysis was also performed in all three modes using the quasi-static axial and torsional stiffness testing results. The results from six cervine specimens, spanning the length range of available specimens, were used for a length-variance optimization analysis. The human cadaver and remaining cervine specimens were excluded from this analysis due to insufficient pre-implantation measurements. The tibia and nail lengths were used as independent factors in an optimization study in Design Expert (10.0, Stat-Ease, Minneapolis, Minn.), with stiffness as the response. The resulting statistical model's ability to predict stiffness was determined using an alpha equal to 0.05. Then, the desirability of a given response (in all three modes) was plotted with respect to the optimization criterion of stiffness being within one standard deviation of the stiffness of the currently in-use Synthes nail stiffness values. Within the Design Expert software, comparisons were made using x² tests with α=0.05 to assess: 1) differences between internal and external quasi-static torsional stiffnesses, and 2) differences between cadaver and cervine tibia-nail constructs for axial stiffness.

Results

Mechanical Testing

Quasi-Static Testing

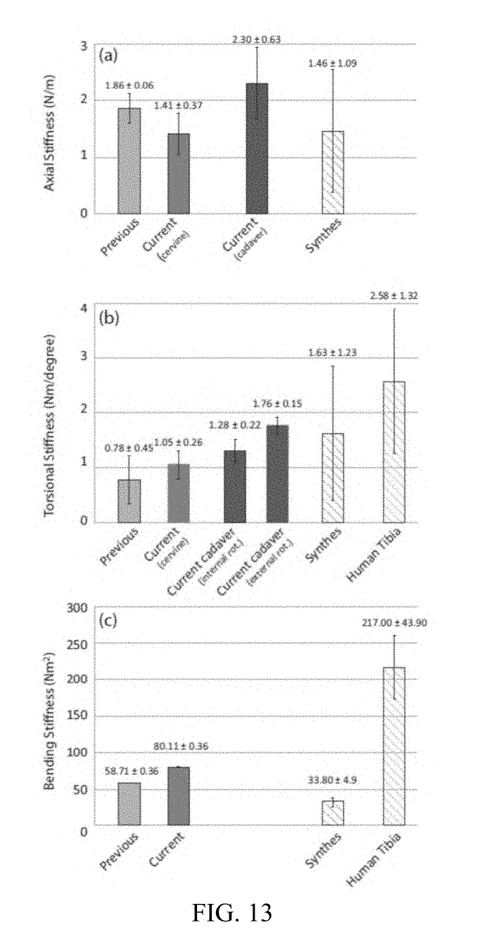

Referring to FIG. 13 is a series of graphs of quasi-static stiffness for two adjustable length orthopedic devices, a prior art orthopedic device, and a human bone. The measured axial of the stainless prototype when inserted into the cervine tibiae with simulated fractures is 1.41±0.37 N/m, and 2.30±0.63 when inserted into fresh-frozen human tibiae. Note that one cervine specimen was excluded from the axial stiffness results due to incomplete data capture. In comparison, a tibial IM nail made by Synthes has an axial stiffness of 1.03 N/m with an interquartile range of 706.2-1326.6 N/m when inserted into formalin-fixed cadaver tibiae with fractures simulated by osteomtomy, and intact cervine tibia stiffness is 2.50 N/m±0.83. The adjustable-length prototype presented in this study was slightly less stiff, but within the same order of magnitude and range of this currently-in-use nail. While axial stiffness is highly variable in studies of intramedullary nails, the prototype performs comparably to previously-reported results. The human cadaver and cervine axial stiffnesses are significantly different (p<0.01). In the torsional mode, the prototype's stiffness when inserted into cervine tibiae was 1.05±0.26 Nm/degree when the average torsional stiffness from both directions of rotation were combined; direction-specific results are given in FIG. 13. This was comparable to the stiffness of the Synthes nail, which was 1.63±1.23 Nm/degree. Intact cadaver tibiae, for comparison, have a reported torsional stiffness of 2.58±1.32 Nm/degree in the human tibia and 3.93 Nm/degree for the cervine tibia. The prototype had an average torsional stiffness of 1.52±0.19 Nm/degree. In quasi-static 4-point bending, the prototype had a stiffness of 80.11±09.360 Nm². The Synthes nail had is reported to have a quasi-static 4-point bending stiffness of 33.80±4.9 Nm². For comparison a human tibia's stiffness is 217±43.9 Nm². In both cyclic axial loading and cyclic 4-point bending, the phase angle did not change markedly over the course of the testing. In axial loading, the phase angle was 0.0±0.314 throughout; that is, peak load and peak displacement occurred simultaneously. (The 0.314 is the error associated with the computation given the loading and sampling rates.) For 4-point bending, the phase angle was exactly zero for all measurements using the stainless steel prototype. The phase angle was exactly zero for most measurements made with the Titanium prototype; the nonzero entries were a phase angle of 0.314 at cycle 1, and 0.079 at cycle 700,000. The consistency of phase angle suggests that minimal, if any, changes in the nail's mechanical behavior occurred during the cyclic loading.

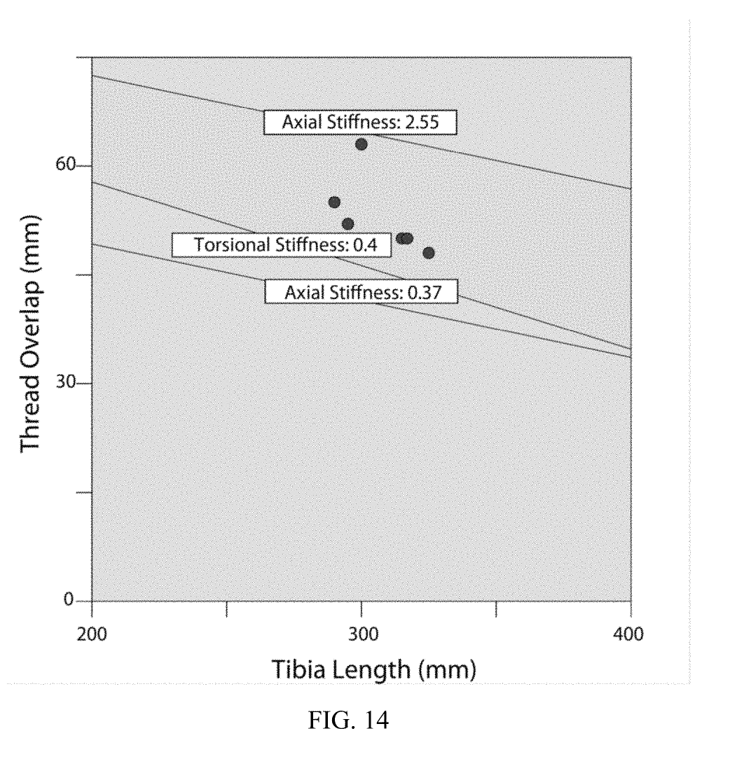

The length variance analysis showed that, as expected, there is a clear relationship between the length of the adjustable nail and its stiffness in all three modes. The models, based on a Pearson's chi-squared test and ANOVA in the Design Expert software, used to predict stiffness were significant (p=0.01 for the axial model, p=0.03 for the torsional model, and p=0.02 for the bending model). This confirms that the expected stiffness of a nail can be determined based on its set length as in FIG. 14. These optimization criteria suggests that any one adjustable-length nail has a 50-60 mm range of length adjustment to always ensure the desired stiffness is met. Outside of this range, optimal stiffness may not be achieved, as suggested by FIG. 14.

Cyclic Loading

In cyclic bending loading, the prototypes survived all 1,000,000 cycles without any visual or aural evidence of failure in both titanium alloy and stainless steel prototypes. No evident damage to or loosening of the locking mechanism was observed. Dynamic bending stiffness differed by only 2.08% from the first cycles to the last in the stainless steel prototype, and by 28.1% in the titanium alloy prototype. The titanium alloy prototype appeared to plastically deform in the form of a visibly apparent small curvature in proximal third of the distal housing (near the middle of the entire nail); rolling it on a flat surface permitted this observation. In axial cyclic loading (when inserted into a human tibia), the prototype survived 103,000 cycles before the distal-most proximal locking screw failed. No evident damage to the locking mechanism was observed, which is particularly noteworthy given that the prototype had already survived the cyclic bending loading tests described above.

Quasi-Static Torsional Failure

In both prototypes, failure in the loosening direction appeared to occur in the distal-most proximal locking screw first. In the tightening direction (that would shorten the overall length of the nail), failure appeared to be in the slipping of the locking nut, which was followed by a re-tightening of the locking nut as additional torsion was applied. The failure loads in torsion in the “tightening” (internal rotation) were 4.33 Nm (stainless steel) and 12.25 Nm (titanium alloy); in the “loosening” (external rotation) direction, they were 2.95 Nm (stainless steel) and 3.10 Nm (titanium alloy). Note that, in both the stainless steel and titanium alloy prototype, the ultimate failure torque was higher in the tightening direction than the loosening direction.

Mechanical Testing

Quasi-Static

The quasi-static stiffnesses of the nail when inserted into deer tibiae compared well to previous prototypes as well as the currently in-use nail. The results of the testing of the titanium alloy prototype, while only one sample, suggest that it is the preferred alloy for future manufacturing of prototypes and, ultimately, adjustable-length nails for use in humans. Its stiffness values are higher than stainless steel in axial and torsional stiffness where the prototype was lower than the Synthes nail, and lower in bending stiffness where the prototype was higher than the Synthes nail. Compared to previous prototypes developed in the laboratory which reported only maximal torsion, the nail is notably stiffer in torsion, regardless of torsion direction; this represents an improvement in the design process. The length variance analysis revealed that any one prototype has approximately 50-60 mm of adjustability before its stiffness may markedly deviate from currently in-use nails. This result suggests that offering different sizes of adjustable nails (such as short, medium, and long) may be necessary to cover the total desired range of adjustability (210-425 mm total length).

Cyclic Loading

The prototype failed by deformation of a locking screw after 103,000 cycles of cyclic axial loading, which occurred after it had survived 1,000,000 cycles of bending load. Future design improvements may include square threads (instead of pointed, as featured in this prototype.) In a similar study, stainless steel locking screws of 4.5 mm and 5 mm failed at 18,238±4009 cycles and 46,736±13,702 cycles respectively. Linear extrapolation of these results suggests that the larger (6 mm diameter) screw would fail at over 100,000 cycles given the same material, thread shape, and surface characteristics, and lends credence to the results. While a single specimen cannot predict the variability of axial failure across a larger sample size, the results suggest that the failure mechanism of the prototype is similar to that of currently-used nails; that is, the locking screws fail first. Additionally, the cyclic bending loading results indicate that under the given conditions, there appears to be no failure or loosening of the locking mechanism. A nail maintaining its structural integrity over 1,000,00 cycles is sufficient per the ASTM standard.

Torsional Failure

The difference between torsional failure in the external and internal rotation directions indicates that the locking nut was not sufficiently tight. To ensure rigid locking, using a torque wrench for tightening may be utilized. The results of torsional failure show that in the direction of tightening, the titanium nail fails at torques that are near the threshold of pain in ankle rotation, and about 25% of ultimate torsional failure of the ankle joint. During healing, this threshold for pain is likely to be much lower, and a compliant patient should not be performing activities which would put unnecessary torsional stresses on the ankle joint. However, in the direction of loosening, the nail fails at torques that could be achieved with much less rotational motion. This suggests that, for maximum torsional stability, the direction of highest torsional resistance should be external rotation. In other words, a right-hand-threaded nail would be appropriate for a right tibia, and a left-hand-threaded for a left tibia. This would ensure that direction of tightening is always in the direction in which a patient is most likely to produce higher torques in actions such as stubbing their toe (external rotation).

### CONCLUSIONS

Within the context of innovations in IM nail technology, the nail design is unique in that it offers a patient-specific fit with a prefabricated device; that is, the device is ready to use, and simply needs to be mechanically locked in length before insertion in the operating room. Length-adjustable nails are available for limb lengthening applications but not for weight-bearing applications. Additional recent innovations in IM nail design have explored different materials. Carbon fiber nails have led to early failure. Composite polymer nails offer a patient-specific fit and have performed well for upper limb bones, but may be unsuitable for the high loads of the lower limb. The IM nail uses the same materials and similar geometry due to those currently inserted into patients, and thus should not experience these problems. An additional recent innovation in mechanical alternatives to the distal locking screws, such as the Talon nail, have proven attractive. Thus, it is possible that the nail-lengthening mechanism might be coupled with an alternative to the distal locking screws for improved performance.

In comparison to other adjustable-length nails, the design presented here is more clinically realistic; the nail is more visually similar to a traditional IM nail and thus more appealing to physicians. Furthermore, the locking mechanism and extendable portion are located in the proximal portion of the bone and are thus distanced from the most common (mid-shaft) fractures.

The unique adjustable-length IM nail successfully meets necessary ASTM standards, and the cyclic bending results further support the case of the mechanical stability of the prototypes, helping to alleviate concerns about stress concentrators that may lead to failure. Both prototypes survived one million cycles of bending, and showed no obvious signs of physical damage or failure of their locking mechanisms. The length variance testing showed that while the stiffness of the nail is dependent on its length, stiffness in all three modes that is comparable to currently in-use nails could be reached using only a few sizes of nails. In addition, these tests revealed an interesting but not unexpected linear relationship between the length of the fractured tibia and the length of nail needed to produce desirable stiffness results. This relationship could be used as in part as a guide and predictor of implant performance.

Length adjustability offers the surgeon a greater chance at guaranteeing a better fit to each patient's intramedullary canal length, without compromising important mechanical attributes. This leads to decreased complication rates, faster recovery times, and overall improved patient welfare. In addition, costs associated with over-stocking of nails are significantly reduced for hospitals. Total manufacturing costs are reduced for device manufacturers, as they no longer need to produce numerous different sizes of IM nails. The implications of a successfully implemented adjustable-length nail make it an exciting clinical innovation, with numerous benefits in many facets of healthcare.

Although the present invention has been described in connection with a preferred embodiment, it should be understood that modifications, alterations, and additions can be made to the invention without departing from the scope of the invention as defined by the claims.

1. An adjustable length orthopedic device, the device comprising:
a shaft having a longitudinal axis extending from a proximal end of the shaft to a distal end of the shaft, the shaft comprising an exterior having a first diameter smaller than the diameter of a medullary cavity of a bone within which at least a portion of the shaft is configured to reside, wherein the shaft comprises at least a portion of an adjustability mechanism located at said proximal end;
a rod comprising a proximal end having at least a portion of an adjustability mechanism complementary to the adjustability mechanism of the shaft, wherein the adjustability mechanism of the shaft and the adjustability mechanism of the rod are configured to engage with one another in an adjustable manner; and
a locking mechanism positioned at the intersection of the shaft and the rod, wherein the locking mechanism is configured to prevent rotation of at least one of the shaft and the rod.

2. The adjustable length orthopedic device of claim 1, wherein the shaft comprises at least one of a threaded interior cavity located at said proximal end and a threaded exterior region located at said proximal end, and wherein the proximal end comprises at least one of a threaded interior cavity and a threaded exterior region, wherein the proximal end of the rod is threaded into or onto the proximal end of the shaft.

3. The adjustable length orthopedic device of claim 1, wherein the device is configured to transit a joint.

4. The adjustable length orthopedic device of claim 1, wherein the locking mechanism comprises a locking nut.

5. The adjustable length orthopedic device of claim 4, wherein the locking nut is movably threaded onto the rod, and wherein a portion of the locking nut is configured to enter the interior cavity of the shaft as the rod is threaded into the shaft.

6. The adjustable length orthopedic device of claim 4, wherein the locking mechanism further comprises a setscrew configured to affix the locking nut in place relative to the rod, or to affix the locking nut in place relative to the shaft.

7. The adjustable length orthopedic device of claim 4, wherein the locking nut is a collet.

8. The adjustable length orthopedic device of claim 7, wherein the collet comprises a tapered region configured to engage a portion of the shaft to interlock the shaft and the rod.

9. The adjustable length orthopedic device of claim 6, wherein the setscrew is a flared setscrew and/or a threaded setscrew.

10. The adjustable length orthopedic device of claim 1, wherein the locking mechanism comprises an interlocking mechanism.

11. The adjustable length orthopedic device of claim 1, wherein the interlocking mechanism comprises a first portion having a biased locking hook and a second portion comprising a locking tab, wherein the biased locking hook is configured to reversibly engage the locking tab to interlock the rod and the shaft.

12. The adjustable length orthopedic device of claim 1, wherein the locking mechanism comprises two locking nuts.

13. The adjustable length orthopedic device of claim 1, wherein the shaft comprises a pivoting locking tab configured to engage the threads of the rod as a locking nut threads onto a portion of the shaft and forces the locking tab to pivot.

14. The adjustable length orthopedic device of claim 1, wherein the device is further configured to attach to another orthopedic device.

15. The adjustable length orthopedic device of claim 1, further comprising a length indicator disposed along at least one of the shaft and the rod.

16. The adjustable length orthopedic device of claim 1, further comprising at least one locking hole located in a distal end of the shaft.

17. The adjustable length orthopedic device of claim 1, further comprising at least one locking hole located in a distal end of the rod.

18. The adjustable length orthopedic device of claim 1, wherein the device is configured for use in an animal other than humans.

19. The adjustable length orthopedic device of claim 1, wherein the locking mechanism is configured to prevent rotation of at least one of the shaft and the rod before the adjustable length orthopedic device is inserted within a bone.

20. The adjustable length orthopedic device of claim 1, wherein the locking mechanism is internal to the rod and/or the shaft.
